(19) United States
(12) Patent Application Publication
(10) Pub. No.: US 2009/0270373 A1
(43) Pub. Date: Oct. 29, 2009

(54) TREATMENT OF DOWN SYNDROM WITH BENZODIAZEPINE RECEPTOR ANTAGONISTS

(75) Inventors: Srinivas Rao, Encinitas, CA (US); Jay Kranzler, La Jolla, CA (US); Jeffery Anderson, San Diego, CA (US)

Correspondence Address: WILSON, SONSINI, GOODRICH & ROSATI, 650 PAGE MILL ROAD, PALO ALTO, CA, 94304-1050, US

(73) Assignee: Cypress Biosciences, Inc., San Diego, CA (US)

(21) Appl. No.: 12/427,430

(22) Filed: Apr. 21, 2009

(30) Foreign Application Priority Data
Apr. 11, 2008 (US) PCT/US2008/060133

(51) Int. Cl. A61K 31/5517; A61P 25/00

(52) U.S. Cl. 514/220; 514/219

(57) ABSTRACT

Pharmaceutical compositions and methods of treating Down Syndrome, mental retardation or both are provided. The pharmaceutical compositions comprise one or more benzodiazepine receptor antagonists, such as flumazenil.

This application is a continuation-in-part filed under 35 U.S.C. § 111 and claims benefit of priority from Serial Number PCT/US2008/060133, filed on Apr. 11, 2008, and designating the United States of America, and from U.S. provisional patent application Ser. No. 60/911,254, filed Apr. 11, 2007, each of which is incorporated by reference herein in its entirety.

### FIELD OF THE INVENTION

This application relates to methods of treating Down Syndrome and other forms of mental retardation with a composition comprising one or more benzodiazepine receptor blocker.

### BACKGROUND OF THE INVENTION

Down Syndrome (trisomy 21) is a genetic disorder caused by the presence of all or part of a third copy of chromosome 21. In addition to various physical characteristics, Down Syndrome is often, though not always, characterized by varying degrees of cognitive impairment—impairment in memory, learning capacity or both. While advances in teaching methods and a trend toward educational mainstreaming has led to an improvement in cognitive development in those who have Down Syndrome, there remain constitutive impairments that cannot be fully addressed through pedagogic methodology alone. In particular, there is a need for improvement in the cognitive abilities of Down Syndrome patients.

Mental retardation is a broader classification of cognitive deficit. A common criterion for diagnosing mental retardation is a score of 70 or below on one or more accepted intelligence quotient (IQ) tests. Mental retardation affects cognitive and motor development. In regards to cognitive development, mental retardation affects learning and memory and especially manifest in slowed acquisition of language skills. As is the case with Down Syndrome, advances in pedagogic methods for those suffering from mental retardation have partially addressed the problems of learning encountered by these individuals. However, there remains a need for the improvement in cognition in those suffering from mental retardation.

Flumazenil is a tricyclic benzodiazepine (8-fluoro-5,6-dihydro-5-methyl-6-oxo-4H-imidazo[1,5-a]benzodiazepine-3-carboxylic acid ethyl ester) that antagonizes (as a competitive inhibitor of) benzodiazepine receptors in the central nervous system. Its preparation is described in U.S. Pat. No. 4,316,839. It has been administered in adults to reverse the effects of benzodiazepines in conscious sedation and general anesthesia. It has also been administered to counteract overdose of benzodiazepine agonists, such as diazepam. Its administration heretofore has primarily been by intravenous injection of an initial injection of about 0.4 mg and follow up doses of 0.2 mg per dose up to a maximum of 1.0 mg. Oral dosing of 30 to 100 mg of flumazenil (also known as Ro 15-1788) in normal adult human subjects has been shown to act as a partial benzodiazepine agonist. (Higgitt et al., “The effects of the benzodiazepine antagonist Ro 15-1788 on psychophysiological performance and subjective measures in normal subjects,” Psychopharmacology, 89 (1986), 396-403.) However, flumazenil effectively antagonizes the effects of diazepam when given orally or intravenously. Id. Thus, flumazenil is often classified as a benzodiazepine receptor antagonist, although its activity is considered in some literature to be mixed (i.e. partial benzodiazepine agonist).

It has recently been shown that use of GABA_{A} antagonists in a murine model of Down Syndrome (Ts65Dn mice) increases memory and declarative learning. F. Fernandez et al., “Pharmacotherapy for cognitive impairment in a mouse model of Down Syndrome,” Nature Neuroscience, Advance Online Publication, (Feb. 25, 2007). Like Down Syndrome patients, Ts65Dn mice demonstrate learning and memory deficits, which are hypothetically due to selective decreases in the numbers of excitatory synapses in the brain rather than gross abnormalities in neuroanatomy. (Id.) Theoretically, triplicate genes found in the Ts65Dn mice shift the optimal balance of excitation and inhibition in the dentate gyrus (and other parts of the brain, perhaps) to a state in which excessive inhibition obscures otherwise normal learning and memory. Thus, enhancement of learning and memory with a GABA_{A} antagonist apparently arises out of antagonizing the GABA_{A} receptor, with concomitant rescue of defective cognition brought about by excessive GABA-mediated suppression of long-term potentiation in the dentate gyrus. Thus, a two-week dosing regimen of 1.0 mg/kg of picrotoxin i.p. showed a clear benefit in the rescue of cognition in Ts65Dn mice. (Id.) In a 4 week crossover study of picrotoxin and bilobalide, both GABA_{A} antagonists demonstrated statistically significant improvement in cognition.

Unfortunately, many GABA_{A} antagonists tend to cause seizure in animal models as well as humans. Thus, there is a need for a non-seizure inducing therapeutic treatment for Down Syndrome, mental retardation or other mental impairment affecting learning, especially declarative learning, memory or both. The present invention meets this need and provides related advantages as well.

### SUMMARY OF THE INVENTION

The foregoing and further needs are met by embodiments of the present invention, which provide a method of treating Down Syndrome or mental retardation, comprising administering to a patient suffering from Down Syndrome or mental retardation an effective amount of a composition comprising at least one active pharmaceutical ingredient selected from a benzodiazepine receptor antagonist, a partial benzodiazepine agonist, or both.

The foregoing and further needs are met by embodiments of the invention, which provide a method of enhancing cognitive function in a patient suffering from mental retardation or Down Syndrome, comprising administering to the patient a cognitive function enhancing amount of an active pharmaceutical ingredient comprising a benzodiazepine receptor antagonist, a partial benzodiazepine agonist or both.

The foregoing and further needs are additionally met by embodiments of the invention, which provide an oral composition for the treatment of mental retardation, Down Syndrome, memory loss or impaired learning, comprising an effective amount of an active pharmaceutical ingredient comprising a benzodiazepine antagonist, a partial benzodiazepine agonist or both.

The foregoing and further needs are met by embodiments of the invention, which provide a sublingual or buccal composition for the treatment of mental retardation, Down Syndrome, memory loss or impaired learning, comprising an effective amount of an active pharmaceutical ingredient comprising a benzodiazepine antagonist, a partial benzodiazepine agonist or both.

Additional characteristics and advantages of the invention will be recognized upon consideration of the following description and the appended claims.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a study protocol in for evaluating the effect of flumazenil on the cognitive function of an animal model of Down syndrome—Ts65Dn mice. Wild type (WT) and Ts65Dn mice were dosed with vehicle, flumazenil or picrotoxin (PTX) and subjected to assays to measure their cognitive function.

FIG. 2A depicts the effects of vehicle, flumazenil and PTX on bodyweights of WT mice over the course of the study.

FIG. 2B depicts the effects of vehicle, flumazenil and PTX on bodyweights of Ts65Dn mice over the course of the study.

FIG. 3A depicts total distance traveled by WT mice in a locomotion assay (LMA) over the course of the study. (Baseline, Days 1, 7, 14, 21 and 28.)

FIG. 3B depicts total distance traveled by Ts65Dn mice in a locomotion assay (LMA) over the course of the study. (Baseline, Days 1, 7, 14, 21 and 28.)

FIG. 4A depicts total distance traveled as a percentage of baseline by WT mice in a locomotion assay (LMA) over the course of the study. (Baseline, Days 1, 7, 14, 21 and 28.)

FIG. 4B depicts total distance traveled as a percentage of baseline by Ts65Dn mice in a locomotion assay (LMA) over the course of the study. (Baseline, Days 1, 7, 14, 21 and 28.)

FIG. 5A depicts marginal distance traveled by WT mice in a locomotion assay (LMA) over the course of the study. (Baseline, Days 1, 7, 14, 21 and 28.)

FIG. 5B depicts marginal distance traveled by Ts65Dn mice in a locomotion assay (LMA) over the course of the study. (Baseline, Days 1, 7, 14, 21 and 28.)

FIG. 6A depicts center distance traveled by WT mice in a locomotion assay (LMA) over the course of the study. (Baseline, Days 1, 7, 14, 21 and 28.)

FIG. 6B depicts center distance traveled by Ts65Dn mice in a locomotion assay (LMA) over the course of the study. (Baseline, Days 1, 7, 14, 21 and 28.)

FIG. 7A depicts the number of vertical rears by WT mice in a locomotion assay (LMA) over the course of the study. (Baseline, Days 1, 7, 14, 21 and 28.)

FIG. 7B depicts the number of vertical rears by Ts65Dn mice in a locomotion assay (LMA) over the course of the study. (Baseline, Days 1, 7, 14, 21 and 28.)

FIG. 8A depicts the percent of vertical rears with respect to baseline by WT mice in a locomotion assay (LMA) over the course of the study. (Baseline, Days 1, 7, 14, 21 and 28.)

FIG. 8B depicts the percent of vertical rears with respect to baseline by Ts65Dn mice in a locomotion assay (LMA) over the course of the study. (Baseline, Days 1, 7, 14, 21 and 28.)

FIG. 9A depicts the baseline amount of time (in seconds) that WT and mutant Ts65Dn mice spent in the margins of the field in a locomotion assay (LMA).

FIG. 9B depicts the baseline amount of time (in seconds) that WT and mutant Ts65Dn mice spent in the center of the field in a locomotion assay (LMA).

FIG. 10A depicts the amount of time (in seconds) that WT and mutant Ts65Dn mice spent in the margins of the field on Day 1 in a locomotion assay (LMA) over the course of the study.

FIG. 10B depicts the amount of time (in seconds) that WT and mutant Ts65Dn mice spent in the center of the field on Day 1 in a locomotion assay (LMA) over the course of the study.

FIG. 11A depicts the amount of time (in seconds) that WT and mutant Ts65Dn mice spent in the margins of the field on Day 7 in a locomotion assay (LMA) over the course of the study.

FIG. 11B depicts the amount of time (in seconds) that WT and mutant Ts65Dn mice spent in the center of the field on Day 7 in a locomotion assay (LMA) over the course of the study.

FIG. 12A depicts the amount of time (in seconds) that WT and mutant Ts65Dn mice spent in the margins of the field on Day 14 in a locomotion assay (LMA) over the course of the study.

FIG. 12B depicts the amount of time (in seconds) that WT and mutant Ts65Dn mice spent in the center of the field on Day 14 in a locomotion assay (LMA) over the course of the study.

FIG. 13A depicts the amount of time (in seconds) that WT and mutant Ts65Dn mice spent in the margins of the field on Day 21 in a locomotion assay (LMA) over the course of the study.

FIG. 13B depicts the amount of time (in seconds) that WT and mutant Ts65Dn mice spent in the center of the field on Day 21 in a locomotion assay (LMA) over the course of the study.

FIG. 14A depicts the amount of time (in seconds) that WT and mutant Ts65Dn mice spent in the margins of the field on Day 28 in a locomotion assay (LMA) over the course of the study.

FIG. 14B depicts the amount of time (in seconds) that WT and mutant Ts65Dn mice spent in the center of the field on Day 28 in a locomotion assay (LMA) over the course of the study.

FIG. 15 shows the baseline discrimination index (DI) for WT and Ts65Dn mice.

FIG. 16 shows the discrimination index (DI) for WT and Ts65Dn mice at the end of Week 1.

FIG. 17 shows the discrimination index (DI) for WT and Ts65Dn mice at the end of Week 2.

FIG. 18 shows the discrimination index (DI) for WT and Ts65Dn mice at the end of Week 4.

### DETAILED DESCRIPTION OF THE INVENTION

The present invention provides methods and pharmaceutical formulations for the enhancement of cognitive functioning, especially memory, learning, or both, especially in individuals suffering from Down Syndrome or mental retardation. The invention provides methods of treating impaired cognitive functioning with one or more active pharmaceutical ingredients selected from chemical entities selected from benzodiazepine receptor antagonists and partial benzodiazepine agonists. Thus, the present invention seeks to improve cognitive functioning—e.g. memory and learning—in individuals whose cognitive functioning has been impaired by a mental disorder that affects their cognition.

Thus, in some embodiments, the invention provides a method of treating Down Syndrome or mental retardation, comprising administering to a patient suffering from Down Syndrome or mental retardation an effective amount of a composition comprising at least one active pharmaceutical ingredient selected from a benzodiazepine receptor antagonist, a partial benzodiazepine agonist, or both. In some embodiments the active pharmaceutical ingredient comprises at least one benzodiazepine receptor antagonist. In some embodiments at least one benzodiazepine receptor antagonist is flumazenil. In some embodiments the method comprises administering to the patient about 0.05 to about 30 mg of flumazenil one to four times daily, preferably about 0.1 to about 15 mg of flumazenil one to four times daily. In some embodiments the active pharmaceutical ingredient comprises at least one partial benzodiazepine agonist. In some embodiments at least one partial benzodiazepine agonist is bretazenil. In some embodiments the method comprises administering about 0.05 to about 30 mg of bretazenil one to four times daily, preferably about 0.1 to about 15 mg of bretazenil one to four times daily. In some embodiments the composition comprising the active pharmaceutical ingredient is an oral, buccal or sublingual composition. In some embodiments the composition comprising the active pharmaceutical ingredient is in the form of a tablet, capsule, gel capsule, caplet or liquid solution or suspension. In some embodiments the composition comprising the active pharmaceutical ingredient is a parenteral preparation. In some embodiments the parenteral preparation is an intravenous injection. In some embodiments the effective amount of the composition comprising the active pharmaceutical ingredient is a sub-seizure inducing amount. In some embodiments the effective amount of the composition is effective to produce a memory enhancing effect, a learning enhancing effect, or both.

Further, in some embodiments, the invention provides a method of enhancing cognitive function in a patient suffering from mental retardation or Down Syndrome, comprising administering to the patient a cognitive function enhancing amount of an active pharmaceutical ingredient comprising a benzodiazepine receptor antagonist, a partial benzodiazepine agonist or both. In some embodiments the active pharmaceutical ingredient comprises at least one benzodiazepine receptor antagonist. In some embodiments at least one benzodiazepine receptor antagonist is flumazenil. In some embodiments the method comprises administering to the patient about 0.05 to about 30 mg of flumazenil one to four times daily, preferably about 0.1 to about 15 mg of flumazenil one to four times daily. In some embodiments active pharmaceutical ingredient comprises at least one partial benzodiazepine agonist. In some embodiments at least one benzodiazepine agonist is bretazenil. In some embodiments the method comprises administering about 0.05 to about 30 mg of bretazenil one to four times daily, preferably about 0.1 to about 15 mg of bretazenil one to four times daily. In some embodiments at least one cognitive function that is enhanced is memory or learning. In some embodiments the composition is an oral, buccal or sublingual composition. In some embodiments the composition comprising the active pharmaceutical ingredient is in the form of a tablet, capsule, gel capsule, caplet, liquid solution, liquid suspension or fast-dissolve tablet. In some embodiments the composition comprising the active pharmaceutical ingredient is a parenteral preparation. In some embodiments the composition comprising the active pharmaceutical ingredient is an intravenous injection. In some embodiments the effective amount of the composition as a sub-seizure inducing amount.

Further, in some embodiments, the invention provides an oral composition for the treatment of mental retardation, Down Syndrome, memory loss or impaired learning, comprising an effective amount of an active pharmaceutical ingredient comprising a benzodiazepine antagonist, a partial benzodiazepine agonist or both. In some embodiments the active pharmaceutical ingredient comprises at least one benzodiazepine receptor antagonist. In some embodiments at least one benzodiazepine receptor antagonist is flumazenil. In some embodiments the composition is in unit dosage form and comprises about 0.05 to about 30 mg of flumazenil, preferably about 0.1 to about 15 mg of flumazenil per dose. In some embodiments the active pharmaceutical ingredient consists essentially of a benzodiazepine antagonist. In some embodiments the benzodiazepine antagonist is flumazenil. In some embodiments the composition is in unit dosage form and comprises about 0.05 to about 30 mg, preferably about 0.1 to about 15 mg of flumazenil per dose. In some embodiments the active pharmaceutical ingredient comprises at least one partial benzodiazepine agonist. In some embodiments at least one partial benzodiazepine agonist is bretazenil. In some embodiments the composition is in unit dosage form an comprises about 0.05 to about 30 mg, preferably about 0.1 to about 15 mg of bretazenil per dose. In some embodiments the active pharmaceutical ingredient consists essentially of a partial benzodiazepine agonist. In some embodiments the partial benzodiazepine agonist is bretazenil. In some embodiments the composition is in unit dosage form an comprises about 0.05 to about 30 mg, preferably about 0.1 to about 15 mg of bretazenil per dose. In some embodiments the form of an oral tablet, caplet, capsule, gel capsule, liquid solution, liquid suspension or fast-dissolve tablet. In some embodiments the composition is in an extended release, delayed release, pulsatile or controlled release dosage form. In some embodiments the effective amount of the active pharmaceutical ingredient is a sub-seizure inducing amount. In some embodiments the effective amount of the active pharmaceutical ingredient is effective to produce a memory enhancing effect, a learning enhancing effect, or both.

Additionally, in some embodiments, the present invention provides a sublingual or buccal composition for the treatment of mental retardation, Down Syndrome, memory loss or impaired learning, comprising an effective amount of an active pharmaceutical ingredient comprising a benzodiazepine antagonist, a partial benzodiazepine agonist or both. In some embodiments the active pharmaceutical ingredient comprises at least one benzodiazepine receptor antagonist. In some embodiments at least one benzodiazepine receptor antagonist is flumazenil. In some embodiments the composition is in unit dosage form and comprises about 0.05 to about 30 mg of flumazenil, preferably about 0.1 to about 15 mg of flumazenil per dose. In some embodiments the active pharmaceutical ingredient consists essentially of a benzodiazepine receptor antagonist. In some embodiments the benzodiazepine receptor antagonist is flumazenil. In some embodiments the composition is in unit dosage form and comprises about 0.05 to about 30 mg, preferably about 0.1 to about 15 mg of flumazenil per dose. In some embodiments the active pharmaceutical ingredient comprises at least one partial benzodiazepine agonist. In some embodiments at least one partial benzodiazepine agonist is bretazenil. In some embodiments the composition is in unit dosage form an comprises about 0.05 to about 30 mg, preferably about 0.1 to about 15 mg of bretazenil per dose. In some embodiments the active pharmaceutical ingredient consists essentially of a partial benzodiazepine agonist. In some embodiments the partial benzodiazepine agonist is bretazenil. In some embodiments the composition is in unit dosage form an comprises about 0.05 to about 30 mg, preferably about 0.1 to about 15 mg of bretazenil per dose. In some embodiments, the buccal or sublingual composition is in the form of a fast-dissolve tablet or strip. In some embodiments, the effective amount of the active pharmaceutical ingredient is a sub-seizure inducing amount. In some embodiments the effective amount of the active pharmaceutical ingredient is effective to produce a memory enhancing effect, a learning enhancing effect, or both.

### Active Pharmaceutical Ingredients (Active Pharmaceutical Agents)

As used herein, the phrase “active pharmaceutical ingredient” (or alternatively “active pharmaceutical agent”) is intended to mean a compound or combination of compounds, at least one of such compounds is a benzodiazepine receptor antagonist or a partial benzodiazepine agonist as described in more detail herein. Thus, unless otherwise limited (e.g. by the delimiters “consisting of” or “consisting essentially of”) recitation of an active pharmaceutical ingredient requires the presence of at least one benzodiazepine receptor antagonist or at least one partial benzodiazepine agonist, but may also include one or more additional pharmaceutical compounds that does not detract from, and in some cases may enhance, the activity of the benzodiazepine receptor antagonist and/or partial benzodiazepine agonist. Thus, combinations of two or more benzodiazepine receptor antagonists, two or more partial benzodiazepine agonists, or at least one benzodiazepine receptor antagonist and at least one partial benzodiazepine agonist (including pharmaceutically acceptable salts, polymorphs, etc.) are included within the scope of the active pharmaceutical ingredient unless otherwise limited.

### Benzodiazepine Receptor Antagonists

As their name implies, benzodiazepine receptor antagonists act on the benzodiazepine receptors on GABA_{A} chloride ion channels to block the effects of GABA. One benzodiazepine receptor antagonist, flumazenil, is used as an antidote to benzodiazepine receptor agonists, such as diazepam and midazolam, in benzodiazepine agonist overdose situations or to counteract the effects of benzodiazepin receptor agonist sedation (e.g. post-operatively).

As discussed in more detail herein, in some embodiments the invention provides oral, buccal and sublingual dosages of benzodiazepine receptor antagonists, such as flumazenil. The oral dosage of flumazenil is expected to be about one to ten, preferably about two to seven times the usual intravenous dose owing to the oral bioavailability of flumazenil, which is approximately 20% of the intravenous bioavailability. Thus, in some embodiments, the invention contemplates administering to the patient one to four doses of flumazenil per day; and those doses, for adults, are expected to be in the range of about 0.05 to about 30 mg, preferably about 0.1 to about 15 mg of flumazenil per dose. For adolescents and pre-adolescents, it is considered that the does will have to be reduced from 2 to 5 fold, depending upon the mass of the patient.

Although flumazenil is a preferred embodiment of the benzodiazepine receptor antagonists of the invention, the person skilled in the art will recognize that other benzodiazepine receptor antagonists, may be used in its place, with appropriate adjustments in dosage made for relative potency, bioavailability and pharmacokinetics.

### Partial Benzodiazepine Agonists

Several partial benzodiazepine agonists are known or under development. These include bretazenil (tert-butyl-(S)-8-bromo-11,12,13,13a-tetrahydro-9-oxo-9H-imidazo[1,5-a]pyrrolo[2,1-c][1,4]benzodiazepine-1-carboxylate (Ro 16-6028)), abecarnil, panadiplon, and imidazenil. Partial benzodiazepine agonists (also known as partial benzodiazepine receptor agonists) partially bind to and activate GABA_{A} chloride channels, but also partially block activation of GABA_{A} channels, thus providing some of the effects of both agonists and antagonists of benzodiazepine receptors.

As discussed in more detail herein, in some embodiments the invention provides oral, buccal and sublingual dosages of partial benzodiazepine agonists, such as bretazenil. The oral dosage of bretazenil is expected to be in the range of about 0.05 to about 30 mg, preferably about 0.1 to about 15 mg of bretazenil per dose. For adolescents and pre-adolescents, it is considered that the does will have to be reduced from 2 to 5 fold, depending upon the mass of the patient.

Although bretazenil is a preferred embodiment of the partial benzodiazepine agonists of the invention, the person skilled in the art will recognize that other partial benzodiazepine agonists may be used in its place.

### Pharmaceutically Acceptable Salts, Stereoisomers, Polymorphs and Hydrates

The person skilled in the art will recognize that various active pharmaceutical ingredients set forth herein are available in free base or salt forms, as enantiomerically pure stereoisomers and/or as polymorphs. Except as otherwise specified herein, recitation of a particular active pharmaceutical ingredient, without any qualification limiting the recitation to the free base or salt, enantiomer or polymorph of the active pharmaceutical ingredient, is intended to incorporate all the pharmaceutically acceptable forms of the active pharmaceutical ingredient, including the free base, pharmaceutically acceptable salts, racemate, enantiomerically pure formulations, amorphous and crystalline forms of the active pharmaceutical ingredient as well as their hydrates.

Examples of pharmaceutically acceptable salts include, but are not limited to, mineral or organic acid salts of basic residues such as amines, and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Conventional non-toxic salts include those derived from inorganic acids such as hydrochloric, hydrobromic, sulfuric, sulfamic, phosphoric and nitric acid; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, benzenesulfonic, toluenesulfonic, methanesulfonic, ethane disulfonic, oxalic and isethionic acids. The pharmaceutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed. (Mack Publishing Company, Easton, Pa., 1985, p. 1418). In the case of flumazenil and bretazenil, the benzodiazepine core has at least one ring amino nitrogen capable of forming a salt with an appropriate acid, such as one of the acids recited above.

Stereoisomers are compounds made up of the same atoms having the same bond order but having different three-dimensional arrangements of atoms which are not interchangeable. The three-dimensional structures are called configurations. Two kinds of stereoisomers include enantiomers and diastereomers. Enantiomers are two stereoisomers which are non-superimposable mirror images of one another. This property of enantiomers is known as chirality. The terms “racemate”, “racemic mixture” or “racemic modification” refer to a mixture of equal parts of enantiomers. The term “chiral center” refers to a carbon atom to which four different groups are attached. The choice of an appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981). Diastereomers are two stereoisomers which are not mirror images but also not superimposable. Diastereoisomers have different physical properties and can be separated from one another easily by taking advantage of these differences.

Different polymorphs of the compounds may also be used. Polymorphs are, by definition, crystals of the same molecule having different physical properties as a result of the order of the molecules in the crystal lattice. The polymorphic behavior of drugs can be of crucial importance in pharmacy and pharmacology. The differences in physical properties exhibited by polymorphs affect pharmaceutical parameters such as storage stability, compressibility and density (important in formulation and product manufacturing), and dissolution rates (an important factor in determining bio-availability). Differences in stability can result from changes in chemical reactivity (e.g. differential oxidation, such that a dosage form discolors more rapidly when comprised of one polymorph than when comprised of another polymorph) or mechanical changes (e.g. tablets crumble on storage as a kinetically favored polymorph converts to thermodynamically more stable polymorph) or both (e.g. tablets of one polymorph are more susceptible to breakdown at high humidity).

### Formulations

The active pharmaceutical ingredients, including pharmaceutically acceptable salts and polymorphic variations thereof, can be formulated as pharmaceutical compositions. Such compositions can be administered orally, buccally, sublingually, intravenously, parenterally, by inhalation spray, rectally, intradermally, transdermally, pulmonary, nasally or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term “parenteral” as used herein includes subcutaneous, intravenous, intramuscular, or intrasternal injection, or infusion techniques. In some preferred embodiments the composition is administered orally, buccally or sublingually; in other preferred embodiments, the composition is administered intravenously.

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

The active pharmaceutical ingredients may be administered per se or in the form of a pharmaceutical composition wherein the active compound(s) is in admixture or mixture with one or more pharmaceutically acceptable ingredients, such as one or more carriers, excipients, disintegrants, glidants, diluents, delayed-release or controlled-release matrices or coatings. Pharmaceutical compositions may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-.beta.-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

The active pharmaceutical ingredients may be complexed with other agents as part of their being pharmaceutically formulated. The pharmaceutical compositions may take the form of, for example, tablets or capsules prepared by conventional means with pharmaceutically acceptable excipients, such as binding agents (e.g., acacia, methylcellulose, sodium carboxymethylcellulose, polyvinylpyrrolidone (Povidone), hydroxypropyl methylcellulose, sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If water-soluble, such formulated complex then may be formulated in an appropriate buffer, for example, phosphate buffered saline or other physiologically compatible solutions. Alternatively, if the resulting complex has poor solubility in aqueous solvents, then it may be formulated with a non-ionic surfactant such as TWEEN™, or polyethylene glycol. Thus, the active pharmaceutical ingredients and their physiologically acceptable solvates may be formulated for administration.

Liquid formulations for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation by the patient.

Delayed release and extended release compositions can be prepared. The delayed release/extended release pharmaceutical compositions can be obtained by complexing drug with a pharmaceutically acceptable ion-exchange resin and coating such complexes. The formulations are coated with a substance that will act as a barrier to control the diffusion of the drug from its core complex into the gastrointestinal fluids. Optionally, the formulation is coated with a film of a polymer which is insoluble in the acid environment of the stomach, and soluble in the basic environment of lower GI tract in order to obtain a final dosage form that releases less than 10% of the drug dose within the stomach.

In addition, combinations of immediate release compositions and delayed release/extended release compositions may be formulated together.

It is anticipated that in some instances it may be advantageous to administer an active pharmaceutical ingredient of the invention as a pulsatile formulation. Such a formulation can be administered as a capsule, tablet or aqueous suspension. For example, a capsule, tablet or aqueous suspension may be formulated containing two or more populations of active pharmaceutical ingredient particle—one containing active pharmaceutical ingredient in an immediate release form (e.g. uncoated or coated with an immediate release coating) and another population in which the active pharmaceutical ingredient is coated with a delayed release coating and/or an enteric coating. In some embodiments, a pulsatile release of active pharmaceutical ingredient results in a longer-lasting formulation, which may be administered on a twice-a-day (b.i.d.) or once-a-day (q.d.) basis. In the case of a capsule, the two populations of particles may be encased within an immediate release or delayed release capsule. In the case of a tablet (including a caplet) the two populations of particles may be compressed, optionally in admixture with an appropriate binder and/or disintegrants, to form a tablet core, which is then coated with an immediate release coating, an enteric coating or both. The tablet then may be coated with a coating that enhances the swallowability of the dosage.

In the case of a liquid suspension, the first population of particles may be uncoated (and indeed wholly or partially dissolved in the aqueous medium) or may be coated with an immediate release coating, an enteric coating or both. The second population of particles is coated with a delayed release coating and optionally an immediate release coating and/or an enteric coating. (Enteric coatings are generally applied where the active pharmaceutical ingredient is sensitive to low pH conditions and thus would be expected to be unstable in the stomach. They may also be applied to the delayed release population of particles in order to add an additional delay to the release of the active pharmaceutical ingredient within the delayed release particles.

In some preferred embodiments, the active pharmaceutical ingredient will be administered as an oral liquid solution or suspension, or as a buccal or sublingual liquid, tablet or gel strip. The person skilled in the art will recognize that buccal and sublingual formulations should be of the fast-dissolving type in order to enhance the ease and convenience of use.

### Treatment of Cognitive Dysfunction

The present invention provides methods of treating cognitive dysfunction, especially the treatment of impaired learning and/or memory associated with Down Syndrome (also referred to as Down's Syndrome or trisomy 21) and/or mental retardation. Down Syndrome is a genetic disorder caused by the presence of all or part of a third copy of chromosome 21. In addition to various physical characteristics, Down Syndrome is often, though not always, characterized by varying degrees of cognitive impairment—e.g. impairment in memory, learning capacity or both. Mental retardation is a broader classification of cognitive deficit. A common criterion for diagnosing mental retardation is a score of 70 or below on one or more accepted intelligence quotient (IQ) tests. Mental retardation affects cognitive and motor development. In regards to cognitive development, mental retardation affects learning and memory and especially manifest in slowed acquisition of language skills.

Benzodiazepine receptor antagonists such as flumazenil inhibit the binding of compounds that bind to the benzodiazepine receptor on GABA_{A} chloride ion channels. The GABA_{A} receptor is a multimeric transmembrane receptor consisting of five subunits arranged around a central ion channel. The GABA_{A} receptors are located within neuronal membranes at a synapse. The ligand GABA (γ-aminobutyric acid) is the endogenous compound that causes this receptor to open. Upon binding of GABA to the GABA_{A} receptor, the receptor changes conformation within the membrane, opening the ion channel, and permitting flow of chloride ions down an electrochemical gradient into the neuron. Because the reversal potential for chloride in most neurons is close to or more negative than the resting membrane potential, activation of GABA_{A} receptors tends to stabilize the resting potential, and can make it more difficult for excitatory neurotransmitters to depolarize the neuron and generate an action potential. The net effect is typically inhibitory, reducing the activity of the neuron.

Benzodiazepine receptors are allosteric ligand binding sites on the GABA_{A} receptor, and are thus separate from the GABA ligand binding sites. Benzodiazepine agonists, such as diazepam, lorazepam and midazolam, bind to benzodiazepine receptors and increase the activity of the chloride channel, thereby enhancing the neuronal activity inhibitory effect of GABA binding to GABA_{A} receptors. The physiologic effect of benzodiazepine agonists is generally sedative and amnesic. In contrast, benzodiazepine receptor antagonists, such as flumazenil, bind to the benzodiazepine receptor and block the effect of benzodiazepine agonists. Thus, flumazenil has been used as an antidote to benzodiazepine agonist overdose or to reverse the sedative effects of benzodiazepine agonist sedatives after surgery. As GABA_{A} antagonists such as picrotoxin rescue cognition in the Ts65Dn murine model of Down Syndrome (See Fernandez, supra), it is considered that benzodiazepine receptor antagonists also counteract the effects of GABA binding—i.e. that they have positive effects on enhancing the activity of neurons involved in cognition. Thus, it is considered that an effective amount of benzodiazepine antagonist would provide cognition enhancing effects, especially to those patients who are genetically disposed to an imbalance in GABA_{A} receptor activity. It is furthermore considered that an effective amount of a benzodiazepine antagonist would provide cognition enhancing effects in patients suffering from cognitive impairment caused by Down Syndrome or mental retardation. As benzodiazepine receptor antagonists are considered to have a lower seizure-inducing potential than GABA_{A} receptor antagonists, such as picrotoxin, it is considered that an effective amount of a benzodiazepine receptor antagonist will also be less than a seizure inducing amount of the benzodiazepine receptor antagonist.

Partial benzodiazepine agonists, such as bretazenil, bind to the benzodiazepine receptor and provide both agonistic and antagonistic effects. Thus, bretazenil has been suggested as an alternative to benzodiazepine antagonists such as diazepam. As GABA_{A} antagonists such as picrotoxin rescue cognition in the Ts65Dn murine model of Down Syndrome, it is considered that partial benzodiazepine agonists, which in part counteract the effects of GABA binding, would also demonstrate positive effects on enhancing the activity of neurons involved in cognition. Thus, it is considered that an effective amount of a partial benzodiazepine agonist would provide cognition enhancing effects, especially to those patients who are genetically disposed to an imbalance in GABA_{A} receptor activity. It is furthermore considered that an effective amount of a partial benzodiazepine agonist would provide cognition enhancing effects in patients suffering from cognitive impairment caused by Down Syndrome or mental retardation. As partial benzodiazepine agonists are considered to have a lower seizure-inducing potential than GABA_{A} receptor antagonists, such as picrotoxin, it is considered that an effective amount of a partial benzodiazepine agonist will also be less than a seizure inducing amount of the partial benzodiazepine agonist.

As the cognitive impairment associated with Down Syndrome and mental retardation may be the result of an imbalance in GABA_{A} functioning, it is considered that a combination of two or more benzodiazepine receptor antagonists, two or more partial benzodiazepine agonists or of at least one benzodiazepine receptor antagonist and at least one partial benzodiazepine agonist may provide an optimal balance of GABA_{A} antagonism and thus optimized improvement in memory, learning or both along.

The present invention provides a method of treating a patient suffering from cognitive impairment, such as a Down Syndrome patient or a patient suffering from mental retardation, comprising administering to the patient an effective amount of an active pharmaceutical ingredient according to the invention. The term active pharmaceutical ingredient is described in more detail above. An “effective amount” of the active pharmaceutical ingredient is an amount of the active pharmaceutical ingredient that provides temporary relief of one or more impairments of cognition. Thus an effective amount of an active pharmaceutical ingredient is expected to provide relief of impaired memory, impaired learning capacity or both. Although the relief provided is considered temporary, the person skilled in the art will recognize that even a temporary improvement in learning capacity can have a long term beneficial effect on long-term learning, as learning tends to be cumulative over time. Thus, the use of the qualifier “temporary” is not intended to exclude potential long-term improvements in cumulative learning.

In some embodiments, the invention provides a method of treating cognitive impairment in a patient, comprising administering an effective amount of an active pharmaceutical ingredient comprising at least one benzodiazepine receptor antagonist, at least one partial benzodiazepine agonist, or both. In some preferred embodiments, the amount of active pharmaceutical ingredient administered to the patient, while being effective to enhance cognition, is a sub-seizure inducing amount. In other words, in preferred embodiments of the invention, the amount of active pharmaceutical ingredient administered to the patient is sufficient to enhance memory, learning or both, but is not sufficient to induce seizure. In some preferred embodiments, the effective amount of flumazenil will be about 0.05 to about 30 mg, preferably about 0.1 to about 15 mg per dose, administered orally, buccally or sublingually 1 to 4 times per day. In some preferred embodiments, the effective amount of bretazenil will be about 0.05 to about 30 mg, preferably about 0.1 to about 15 mg per dose, administered orally, buccally or sublingually 1 to 4 times per day.

### Example 1

### The Effect of Flumazenil on Ts65Dn Mice

The effect of a benzodiazepine receptor antagonist, flumazenil, on a murine model of Down Syndrome is investigated using Ts65Dn mice. The validity of the Ts65Dn mouse as a model of the cognitive impairments associated with Down Syndrome is established by Fernandez et al., supra.

A 4-week longitudinal crossover study is carried out following the method outlined by Fernandez et al., supra. Wild-type and Ts65Dn mice (3-4 months of age) are randomly assigned to groups receiving daily i.p. injections of saline or flumazenil (1.0 mg/kg), and are submitted to four weekly repetitions of object recognition testing, in which the animals are serially presented with four different object sets. At the 2-week midpoint of the experimental period, wild-type and Ts65Dn mice that have been receiving saline are randomly segregated into groups that either continue to receive daily saline injections or begin to receive daily injections of flumazenil. Conversely, wild-type and Ts65Dn mice that have been chronically administered flumazenil in the first 2 weeks of testing are switched onto a saline regimen. Alongside saline and flumazenil, bilobalide (i.p. 5.0 mg/kg) may be evaluated as a positive control. Bilobalide is a picrotoxin-like compound that may safely be administered for the whole 4-week experiment.

During the evaluation Ts65Dn and wild-type mice are tested for novel object recognition. Ts65Dn mice treated with flumazenil during the first or second 2 week period have normalized object recognition performance as do those treated with bilobalide throughout the study.

Flumazenil may also be tested for its effects on declarative memory in the novel object recognition test and in a modified spontaneous alternation task. Wild-type and Ts65Dn mice may be administered flumazenil (3 mg/kg in milk via voluntary oral feeding or 1 mg/kg i.p.). The wild-type and Ts65Dn mice are administered from 5 to 30 doses of milk or milk-flumazenil (or saline or flumazenil solution i.p.). The mice are then subjected to two repetitions of novel object recognition testing or three daily T-maze sessions at the tail end of the treatment regimen. It is expected that milk (or saline) treated Ts65Dn mice will show an inability to object novelty in the object recognition task, whereas the flumazenil-tested Ts65Dn mice will show discrimination indices on a par with those of wild-type mice. In the spontaneous alternation task, milk fed (saline i.p.) Ts65Dn mice will show a pattern of impairment similar to untreated Ts65Dn mice, whereas flumazenil treated Ts65Dn mice will show normal levels of alternation. Comparison of treated and untreated Ts65Dn mice will provide controls for any possible arm bias in the spontaneous alternation task.

It is further expected that flumazenil-treated Ts65Dn mice will show long-term improvement in novel object recognition testing (up to at least 2 months after treatment) when treated with flumazenil for at least about 15 days.

Since the ability of animals to learn and remember is thought to be encoded at the synaptic level, and involves the ability of synapses to undergo long-term changes in synapse strength, long-term potentiation (LTP) in the dentate gyrus may be evaluated. Normalized LTP in the dentate gyrus of the flumazenil treated Ts65Dn mice about 1 month after cessation of drug treatment demonstrates long-term improvement in rescue of synapse performance related to memory and learning.

### Example 1A

### Assessment of Potential Cognitive Enhancing Effects of Flumazenil in a Mouse Model of Down Syndrome

Study Protocol: Subjects

Segmental trisomy 16 (Ts65Dn) mice along with WT controls were obtained from the Jackson Laboratories (Bar Harbor, Me.). The mice are derived by mating female carriers of the 1716 chromosome (B6EiC3H-a/ATs65Dn) with (C57BL/6JEi×C3H/HeJ)F1 (JAX # JR1875) males. The Ts65Dn mice are maintained on the B6/C3H background (Jackson Laboratories product information).

Upon receipt at the University of New England animal facility, the WT and Ts65Dn mice were housed in standard Plexiglas cages and kept on a 12 h/12 h light-dark cycle (lights on 0700 h). Mice were shipped either singly or in cohorts up to four/carrier. These groupings were maintained at the UNE facility whenever possible (the exception was when fighting of group house mice required separation of an aggressor to a separate cage.

Food and water were available ad libitum with the exception of the formal testing procedures when the mice were in the LMA or object recognition chambers). Animals were maintained under standard housing conditions (temperature 22±2° C.) and relative humidity between 40-70%). Mice were acclimated to these controlled housing conditions for at least three weeks before inclusion in any behavioral studies.

All experimental procedures were approved by the University of New England Institutional Animal Care and Use Committee (IACUC), and were conducted in compliance with the NIH Guide for the Care and Use of Laboratory Animals.

Study Protocol: General Overview

Drug Treatments

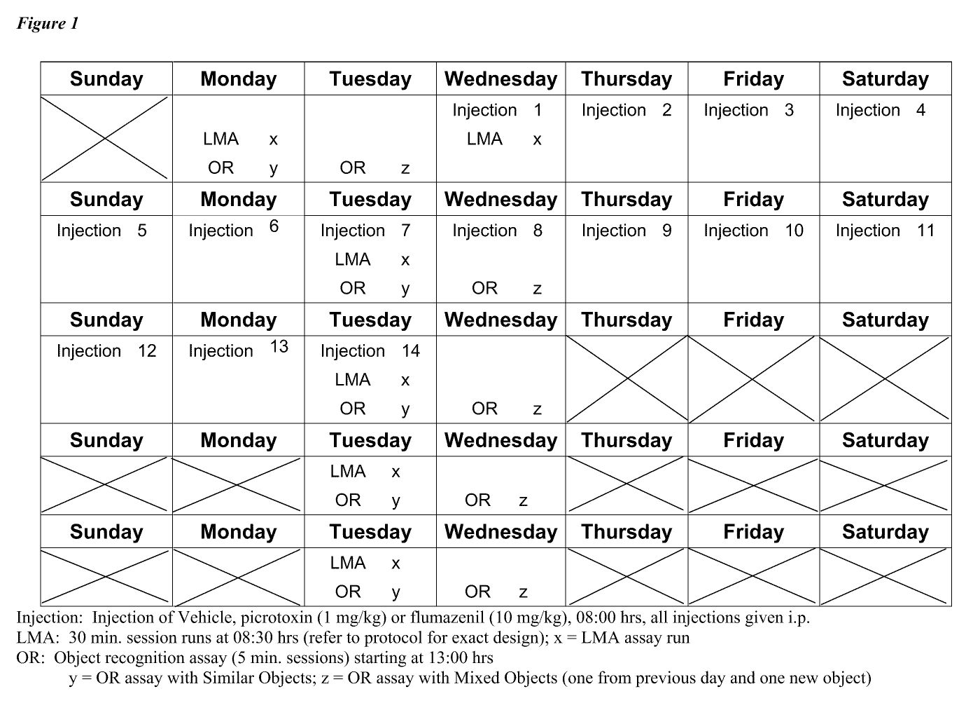

Following baseline determinations and group assignments, WT and Ts65Dn mice received intraperitoneal (i.p.) injections once/day (08:00) for the two-week duration of the drug treatment regimen (FIG. 1). Drugs were dissolved in physiological saline. Two drops of Tween 80 were used to facilitate solution formation for flumazenil. Picrotoxin was initially made up at 50× concentration in 20% ethanol and then diluted down to a 1 mg/ml solution with 0.4% ETOH in saline. Doses of drugs were based on previous behavioral research in mice (1 mg/kg for PTX and 10 mg/kg for flumazenil). Injections were given in a volume of 10 ml/kg. A vehicle control was run along side the treatment groups (0.4% ETOH in saline).

Experimental Protocol

FIG. 1 graphically depicts the protocol. After habituation to the animal facility, mice were tested for baseline behaviors in the open-field locomotor assay (LMA)(30 min session) and the novel object recognition assay. Primary experimental groups were divided by genotype (Ts65Dn mice and wild-type control littermates). These two groups were further divided into drug treatments (vehicle, PTX and flumazenil) with a targeted n-size of 8 mice/group (24 WT and 24 Ts65Dn mice total). The group assignments were made in a semi-random process due to constraints in animal availability (staggered shipping) and choice of initial treatment groups (e-mail documentation between Bilsky laboratory and Cypress Bioscience on file). Daily morning injections began on Day 1 and went through the morning of Day 14. On Days 7/8 and 14/15 (13:00) mice were tested in the behavioral assay. Mice were kept for an additional two weeks and re-tested on Days 21/22 and 28/29 to assess the duration of the drug treatment effect.

Study Overview: Open Field Assay

General levels of arousal, locomotor activity and anxiety were assessed using an automated open-field activity monitoring system (Coulbourn Instruments with TruScan software). The open field test that were run were fully automated assays that quantified many different aspects and patterns of movement, including total distance traveled, time spent moving, rearing, and various stereotypic patterns of activity. The software calculates the time spent in the middle portions of the open field versus the outer zones alongside the outside walls. These variables can be used as one measure of general anxiety levels.

Mice were placed into an open field (26 cm×26 cm) in a brightly lit room. Two sensor rings (x-y plane and a vertical z plane) generate an array of infrared beams that surround the chamber. The pattern of beam breaks was recorded and the data fed into a personal computer. The TruScan software interpreted the beam breaks to calculate the movement variables and patterns of activity, with a temporal resolution of 100 msec. A 30 minutes session was used for assessment of the mice on Day 0 (Baseline) and then on Days 1, 7, 14, 21 and 28. Data were exported into an Excel spreadsheet, graphed and analyzed using parametric and non-parametric statistics.

Study Protocol: Novel Object Recognition

The novel object recognition task is based on the innate tendency of rodents to differentially explore novel objects over familiar ones. The task is well validated for evaluating rodent memory and the effects of drug treatments on learning/memory performance.

Mice were trained and tested once per week, each experimental session separated by a one-week interval. Mice were submitted to daily handling sessions, and given an opportunity to habituate to an open field arena (48 cm×48 cm wide×25 cm), where they were exposed to two different objects (complex protocol), during a 5-minute training session. Objects were made from various non-porous materials that differ in shape and color. Mice were unable to climb onto the objects and the objects were generally consistent in height and volume, and were roughly symmetrical on a horizontal plane. Objects were positioned in two corners of the apparatus. To control for odor cues, the open field arena and the objects are thoroughly cleaned with 90% ethanol, dried, and ventilated for a few minutes between mice.

A 5-minute testing session was conducted 24 h after training. Here, the mice were presented with the object they had explored the previous day, and a new item (the objects being alternatively positioned in one corner or another in a balanced fashion within a given week, and from one week to another). Memory was operationally defined as the proportion of time animals spent investigating the novel object minus the proportion spent investigating the familiar one [Discrimination Index, DI=(Novel Object Exploration Time/Total Exploration Time)−(Familiar Object Exploration Time/Total Exploration Time)×100], where exploration constituted any investigative behavior (i.e., head orientation, sniffing occurring within <1.0 cm) or deliberate contact that occurred with each object. All behavioral sessions were videotaped and reviewed by trained observers who are blinded to the drug treatments (genotype was difficult to blind with the strain of mice due to Ts65Dn facial malformations).

The overall study protocol is depicted in FIG. 1, where days on which injections (Vehicle, picrotoxin (1 mg/kg), or flumazenil (10 mg/kg)) are administered are indicated by a number (1, 2, 3 . . . 14), indicating the number of the day of the study, days on which LMA assay is performed are indicated with an “x”, days on which the object recognition assay (OR) is performed with similar objects are indicated with a “y” and days on which the OR is performed with different objects (one new and one from the day before) are indicated with a “z”.

Results

The following paragraphs present the results of the study.

Bodyweights

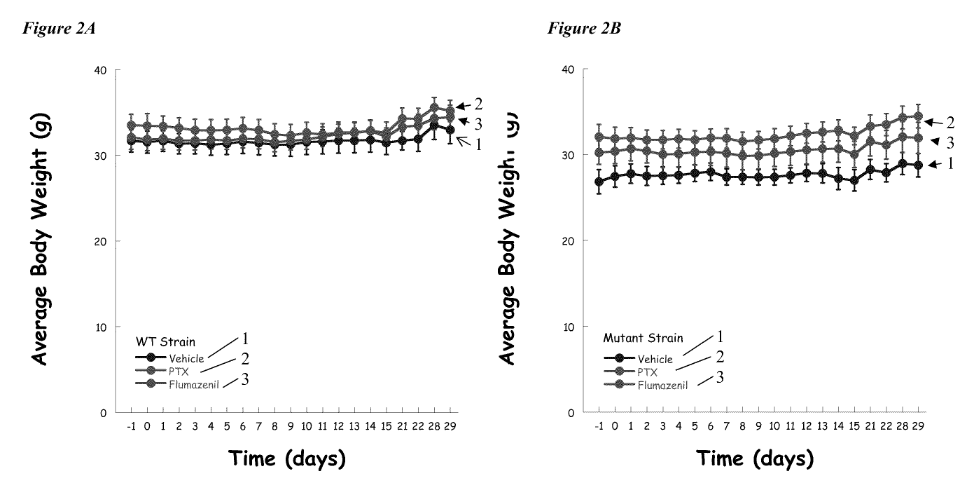

As shown in FIGS. 2A and 2B: Adult male mice (˜4 months old) were used for all studies and weights remained relatively stable across the 28+ days of the protocol. In the figures, “WT” indicates wild type (i.e. non-mutant) mice, whereas “mutant strain” refers to Ts65Dn mice. No major effects of the drug treatments were noted on either WT or mutant strain mice. The slight increase in body weight (BW) was seen following the 14 day injection/testing protocol which may reflect a decrease in stress associated with the repeated daily handling. BWs were taken each day of the injection/testing phase (baseline through Day 15, and then only on Days 21/22 and 28/29).

LMA (Total Distance Traveled)

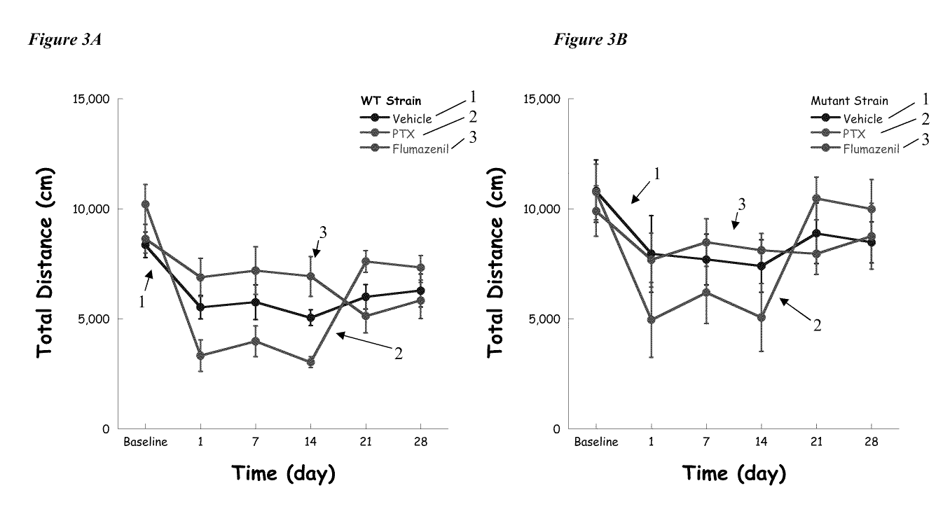

FIGS. 3A and 3B depict baseline comparisons on distance traveled in a 30 minute open field session. The baseline comparisons indicated that there were no major differences between WT and mutant strains. Informal observations noted a more impulse-like movement pattern with the mutants (spurts of forward locomotion). Semi-random group assignments led to all treatment groups being roughly equal at baseline. There was mild habituation across the 6 LMA sessions, with the biggest drop occurring between Baseline and Day 1. Note that this is to be expected given the short interval between the baseline and Day 1 testing, whereas the other points are separated by a week). The picrotoxin (PTX) dosing produced a modest decrease in LMA activity during the dosing phase (Days 1, 7 and 14). The effects were gone within 7 days of the last dosing being administered.

LMA (Percent Baseline DT)

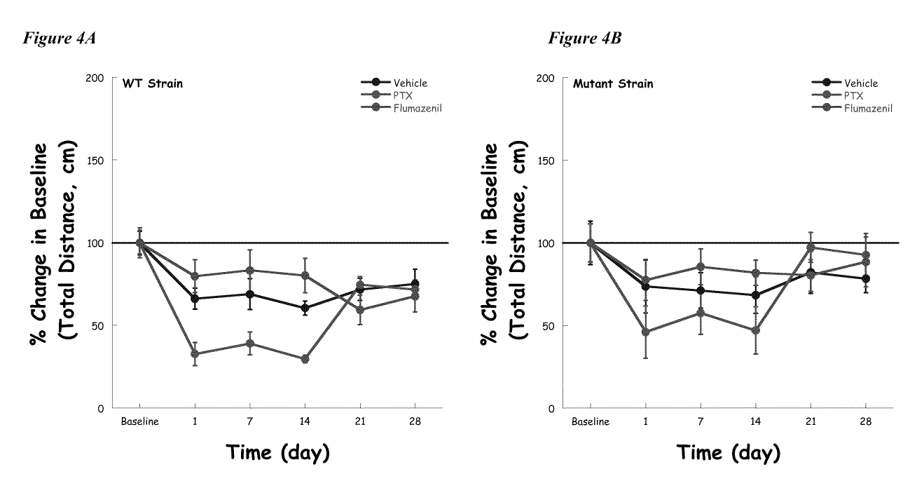

FIGS. 4A and 4B depict data from FIGS. 3A and 3B, respectively, re-graphed as percent of baselines for each experimental group. The effects of PTX appear more pronounced in these figures.

LMA (Marginal Distance)

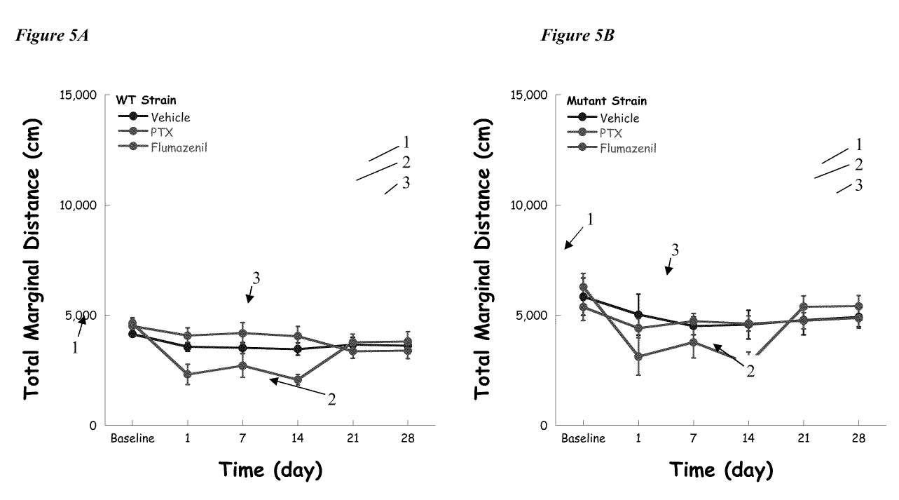

FIGS. 5A and 5B show baseline comparisons on marginal distance traveled by WT and mutant strain mice, respectively. The baseline comparisons indicated no significant differences between treatment groups for WT or mutant strains. There was very little habituation across sessions in the vehicle or flumazenil treated animals for either strain. The PTX treatment effect (decreased distance traveled) was seen in the in this analysis, but is ascribed to a general decrease in activity rather than a change in anxiety levels.

LMA (Center Distance)

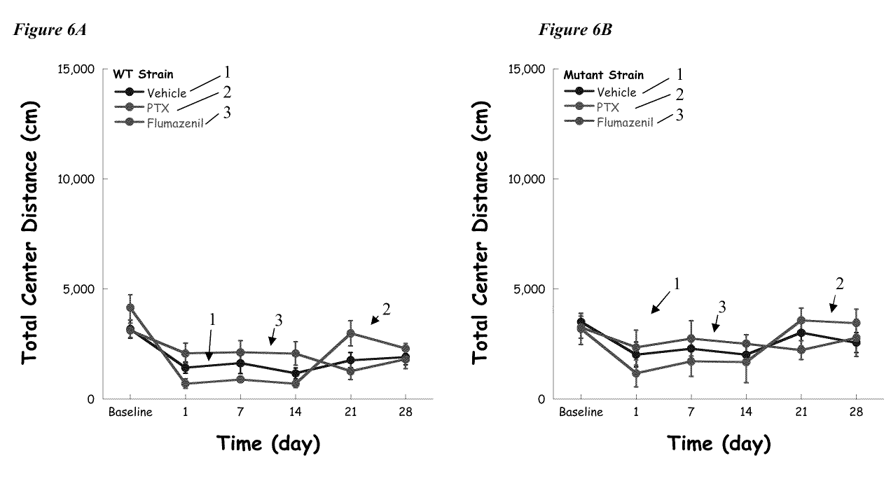

FIGS. 6A and 6B show the center distance data for WT and mutant strain mice, respectively. The center distance data parallel the margin distance data of the previous figure. As expected, baseline levels of locomotor activity were lower than margin activity for both WT and mutant mice. No major differences were seen between treatment groups at baseline. The PTX group again showed decreased levels of activity during the drug administration phase of the experiments (Days 1, 7 and 14) with a mild rebound effect 7 and 14 days post dosing. Flumazenil effects were similar to vehicle, and were generally stable across sessions with the exception of minor habituation after the first exposure to the chambers.

LMA (Vertical Rearing)

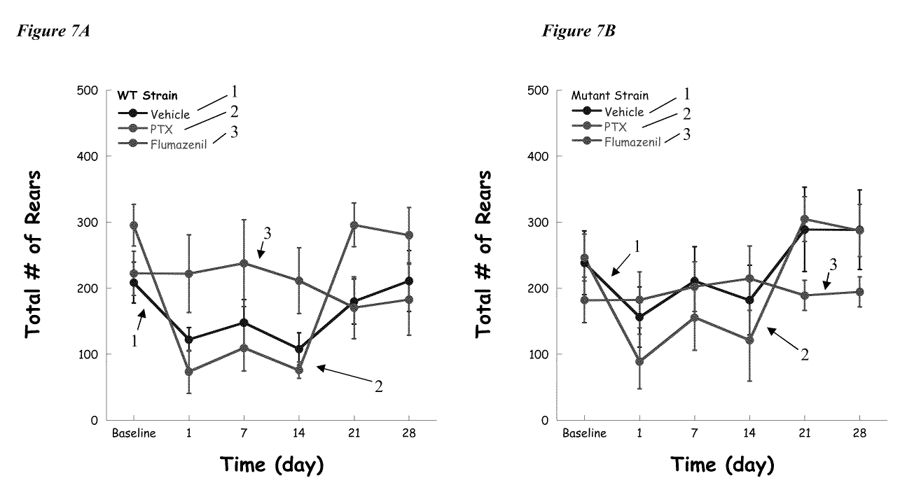

FIGS. 7A and 7B demonstrate that vertical rearing behavior was more variable than the other measures of locomotor activity (time and distance). In general, the two strains had similar levels of rearing activity during the baseline session. The largest effect was seen with the PTX group, with both WT and mutants dropping significantly during the drug administration phases. Interestingly, the flumazenil group showed the least effect over the course of the 4 week testing regimen.

LMA (Percent Baseline VR)

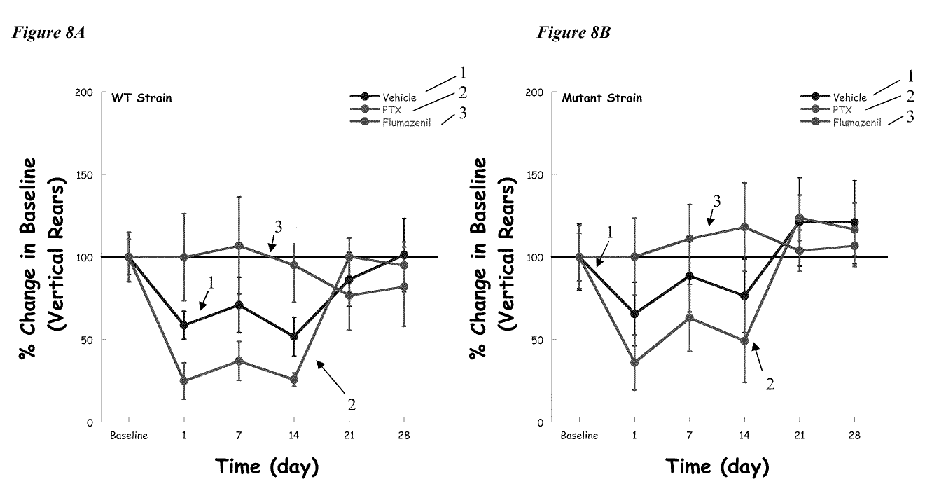

In FIGS. 8A and 8B, data from FIGS. 7A and 7B, respectively, are re-graphed as percent of baselines for each experimental group. The effect of PTX can be more clearly seen in this graph (Days 1, 7 and 14).

LMA (Margin vs. Center Time) (Baseline)

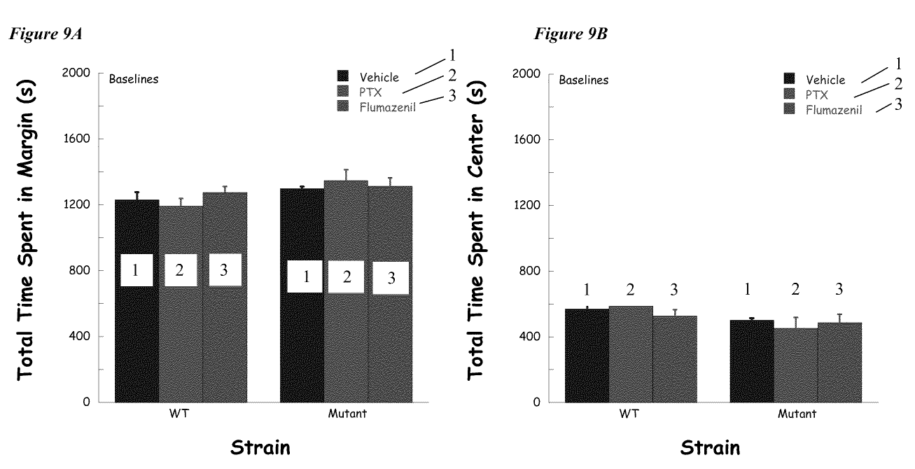

As shown in FIGS. 9A and 9B, both WT (9A) and mutant strain (9B) mice spent significantly more time in the margins versus the center zones of the open field at baseline. There were no differences between the WT and mutant mice in terms of preferences and no differences between the three group assignments.

LMA (Margin vs. Center Time) (Day 1)

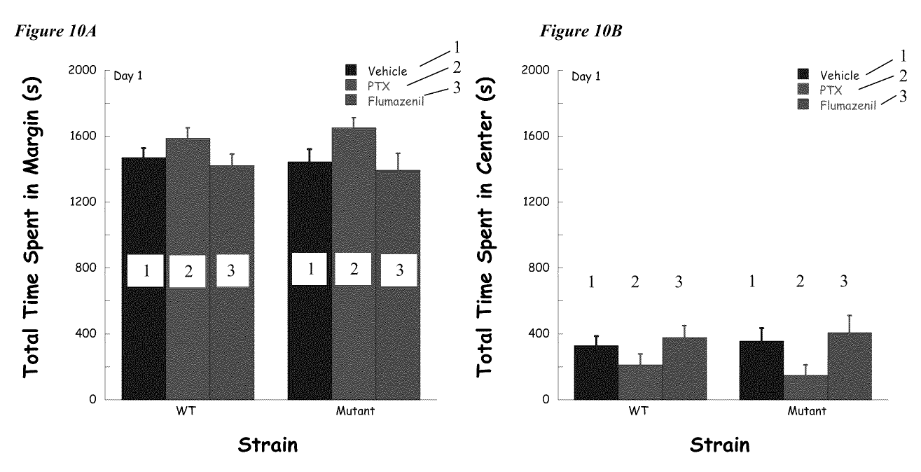

FIGS. 10A and 10B show that on Day 1 of drug treatment, no major strain or drug treatment effects are noted. There was a trend for decreased center time in the PTX treated animals.

LMA (Margin vs. Center Time) (Day 7)

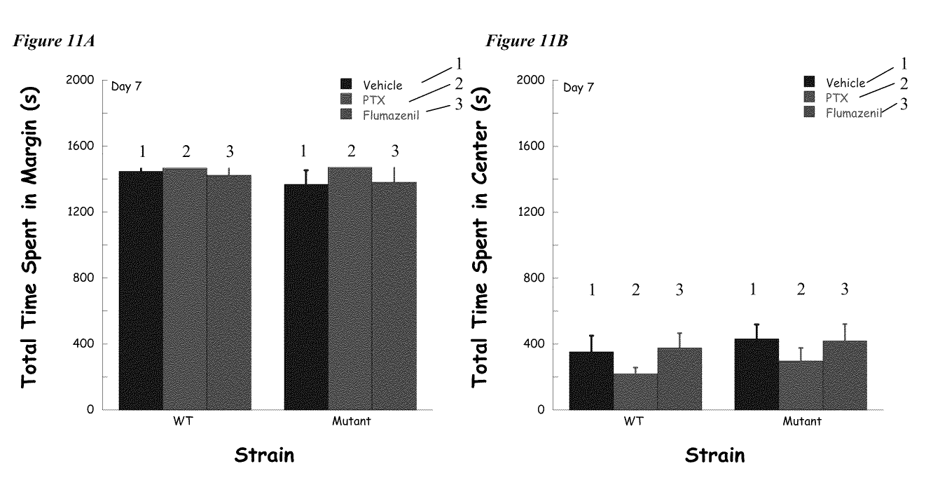

FIGS. 11A and 11B show that the Day 7 data are similar to the Day 1 data, with a small decrease in time spent in the center zone noted for the PTX treated mice.

LMA (Margin vs. Center Time) (Day 14)

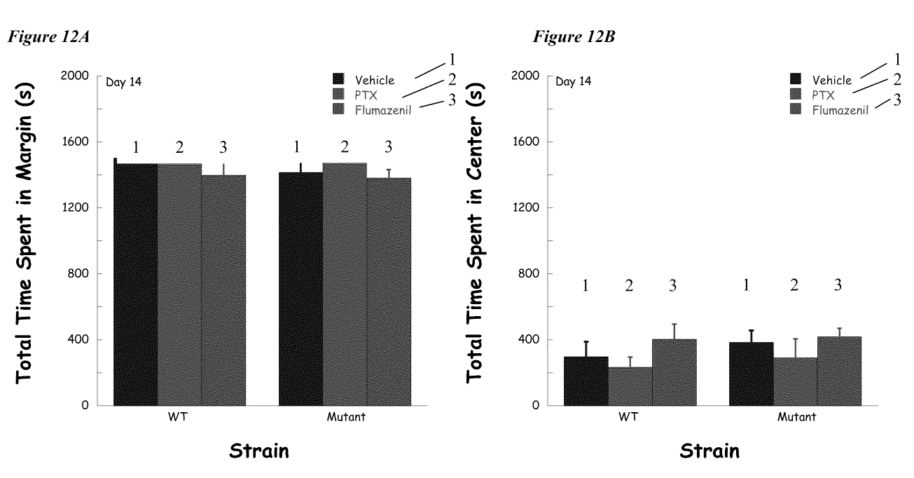

FIGS. 12A and 12B show that there were no major effects of strain or drug treatment noted on Day 14.

LMA (Margin vs. Center Time) (Day 21)

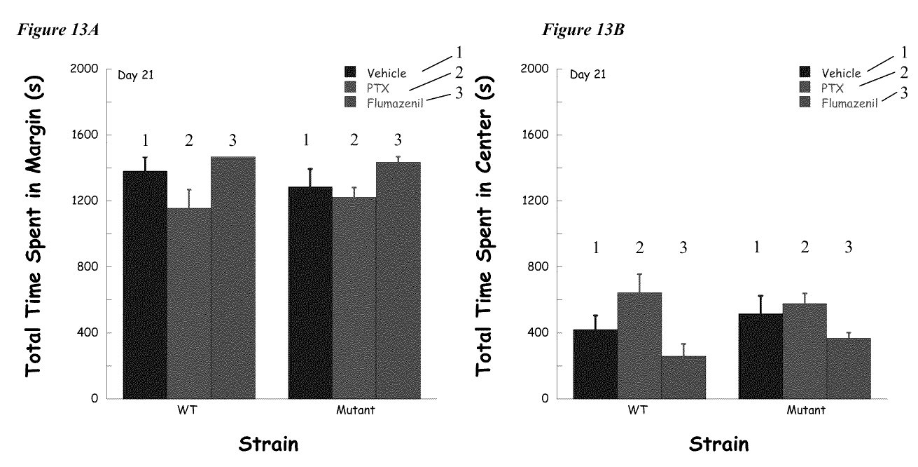

FIGS. 13A and 13B show that no strain effects were seen between the vehicle controls of the WT or mutant mice. It is interesting to note the reversal of the trend in PTX treated mice (now increased time spent in center during the washout period). This rebound effect was also seen in some of the other LMA graphs.

LMA (Margin vs. Center Time) (Day 28)

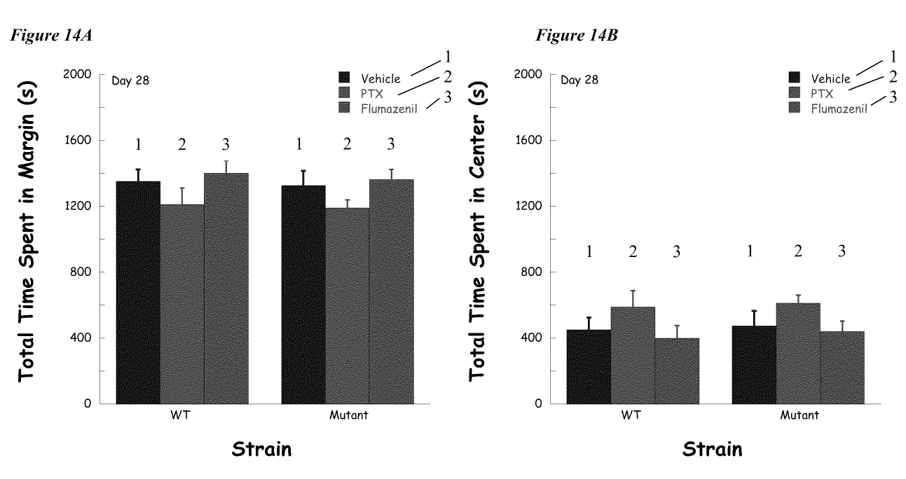

FIGS. 14A and 14B show that data for Day 28 are similar to the Day 21 data in FIGS. 13A and 13B, respectively. The trend for increased time spent in the center zone for PTX treated mice is again noted.

Discrimination Index (Baseline)

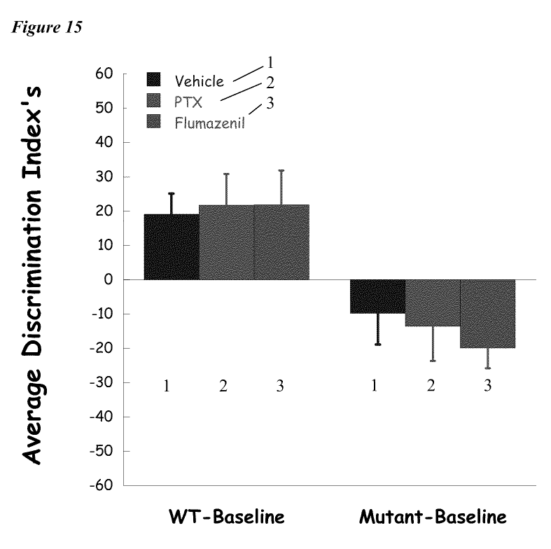

FIG. 15 shows that the WT control mice for each group assignment were able to discriminate the novel object as indexed by the positive DI numbers (see slide 6). In contrast, the Down syndrome mutant mice had negative DI values, indicating a dysfunctional learning/memory process.

Discrimination Index (Week 1)

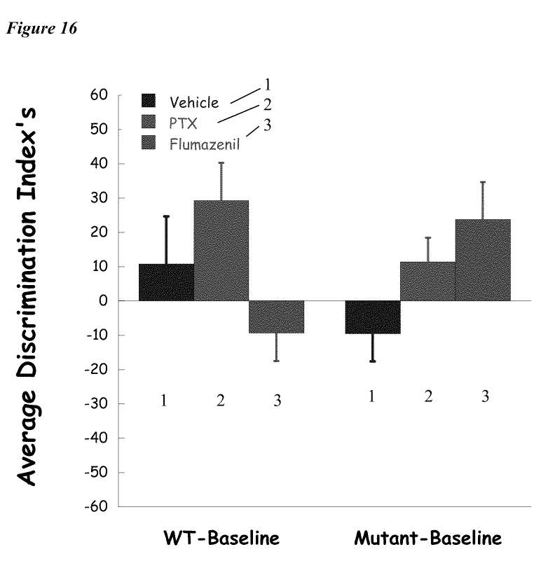

FIG. 16 shows that after 1 week of drug therapy, the WT vehicle treated mice stayed positive with respect to DI, and the PTX treatment increased DI values to a mean of almost 30. Flumazenil may have interfered with learning and memory in the WT mice at the doses administered (inverted U shaped curve). The vehicle treated mutant mice had a negative DI. Both PTX and flumazenil had positive effects at the doses tested.

Discrimination Index (Week 2)

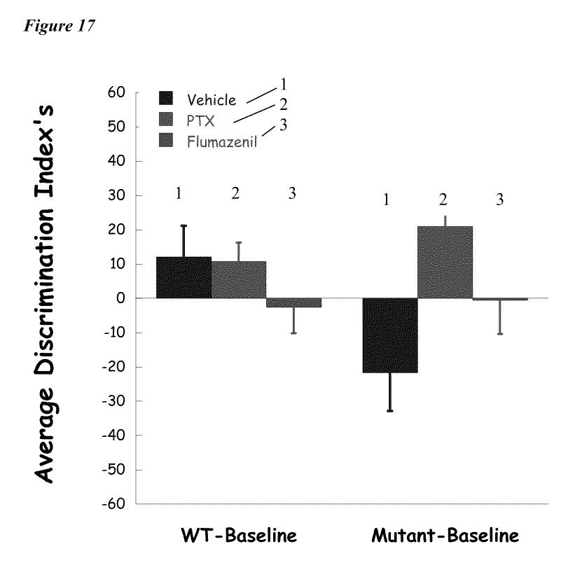

FIG. 17 shows that after 2 weeks of drug therapy, the WT vehicle treated mice stayed positive with respect to DI. There were no differences between the vehicle treated animals and the PTX and flumazenil treated WT mice (though a trend for decreased DI is still noted with flumazenil). The vehicle treated mutant mice had a negative DI. Both PTX and flumazenil had positive effects at the doses tested (the PTX effect was significantly greater).

Discrimination Index (Week 4)

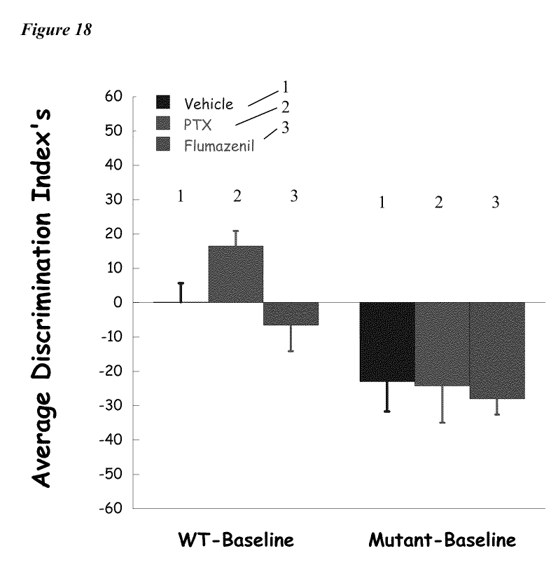

As can be seen in FIG. 18, by 2 weeks post dosing, the WT vehicle treated mice had a DI of ˜0. (This may reflect the effects of continued exposure to the test apparatus and general habituation to the chamber/procedure.) The PTX WT mice still exhibited a positive DI and the flumazenil WT mice had a slight negative DI similar to weeks 1 and 2. All three treatment groups for the mutant mice had robust negative DI values indicating that any positive drug effects had disappeared during the washout period.

Conclusion

As can be seen in the foregoing description of the results, and in the appended figures, the treatment of Ts65Dn mice, a recognized animal model of Down syndrome, flumazenil had a beneficial effect in the object recognition assay as compared to vehicle. The inventors interpret these results as evidence supporting flumazenil's efficacy in the treatment of Down syndrome.

### Example 2

### The Effect of Bretazenil on Ts65Dn Mice

The effect of a benzodiazepine receptor antagonist, bretazenil, on a murine model of Down Syndrome is investigated using Ts65Dn mice. The validity of the Ts65Dn mouse as a model of the cognitive impairments associated with Down Syndrome is established by Fernandez et al., supra.

A 4-week longitudinal crossover study is carried out following the method outlined by Fernandez et al., supra. Wild-type and Ts65Dn mice (3-4 months of age) are randomly assigned to groups receiving daily i.p. injections of saline or bretazenil (1.0 mg/kg), and are submitted to four weekly repetitions of object recognition testing, in which the animals are serially presented with four different object sets. At the 2-week midpoint of the experimental period, wild-type and Ts65Dn mice that have been receiving saline are randomly segregated into groups that either continue to receive daily saline injections or begin to receive daily injections of bretazenil. Conversely, wild-type and Ts65Dn mice that have been chronically administered bretazenil in the first 2 weeks of testing are switched onto a saline regimen. Alongside saline and bretazenil, bilobalide (i.p. 5.0 mg/kg) may be evaluated as a positive control. Bilobalide is a picrotoxin-like compound that may safely be administered for the whole 4-week experiment.

During the evaluation Ts65Dn and wild-type mice are tested for novel object recognition. Ts65Dn mice treated with bretazenil during the first or second 2 week period have normalized object recognition performance as do those treated with bilobalide throughout the study.

Bretazenil may also be tested for its effects on declarative memory in the novel object recognition test and in a modified spontaneous alternation task. Wild-type and Ts65Dn mice may be administered bretazenil (3 mg/kg in milk via voluntary oral feeding or 1 mg/kg i.p.). The wild-type and Ts65Dn mice are administered from 5 to 30 doses of milk or milk-flumazenil (or saline or flumazenil solution i.p.). The mice are then subjected to two repetitions of novel object recognition testing or three daily T-maze sessions at the tail end of the treatment regimen. It is expected that milk (or saline) treated Ts65Dn mice will show an inability to object novelty in the object recognition task, whereas the flumazenil-tested Ts65Dn mice will show discrimination indices on a par with those of wild-type mice. In the spontaneous alternation task, milk fed (saline i.p.) Ts65Dn mice will show a pattern of impairment similar to untreated Ts65Dn mice, whereas flumazenil treated Ts65Dn mice will show normal levels of alternation. Comparison of treated and untreated Ts65Dn mice will provide controls for any possible arm bias in the spontaneous alternation task.

It is further expected that flumazenil-treated Ts65Dn mice will show long-term improvement in novel object recognition testing (up to at least 2 months after treatment) when treated with flumazenil for at least about 15 days.

Since the ability of animals to learn and remember is thought to be encoded at the synaptic level, and involves the ability of synapses to undergo long-term changes in synapse strength, long-term potentiation (LTP) in the dentate gyrus may be evaluated. Normalized LTP in the dentate gyrus of the bretazenil treated Ts65Dn mice about 1 month after cessation of drug treatment demonstrates long-term improvement in rescue of synapse performance related to memory and learning.

The foregoing experiment may be repeated with one or more other benzodiazepine receptor antagonists and/or partial benzodiazepine agonists.

### Example 3

### Effect of Flumazenil and Bretazenil on Cognition in Down Syndrome Patients

Fifteen adult or adolescent Down Syndrome patients experiencing at least some level of cognitive impairment participate in a double-blind, cross-over comparison of four treatments. The drugs administered are flumazenil (5 and 20 mg), bretazenil (5 and 20 mg) or placebo. Subjects are randomly assigned to treatments according to a Williams Square design (Higgitt, supra (citing Williams, “Experimental designs balanced for the estimation of residual effects of treatments, Aust. J. Sci. Res. 2:149-168 (1949))). Each subject receives a different sequence of four treatments balanced for carry-over effects and separated by at least 1 week of wash-out period. At each treatment session, subjects are tested before treatment and at time points 30, 60, 90, 120, 150 and 180 min. after drug administration on a set of psychophysiological measures. At pretest, 60, 120 and 180 min., paper and pencil performance measures and subjective ratings are administered. (Id. (citing Karniol et al., “Comparative psychotropic effects of trazadone, imipramine and diazepam in normal subjects,” Curr. Ther. Res. 20 (1976), 337-347.

Psychophysiological indices to be measured include: EEG, skin conductance, finger tremor, critical flicker fusion threshold, blood pressure and pulse rate, key tapping rate, and reaction time. Paper and pencil performance measures include: A cancellation task, digit symbol substitution and a symbol copying test. Self ratings include mood ratings, and a bodily symptom scale.

EEG and evoked responses: These are recorded during the same EEG recording procedure. Recordings are made from bipolar electrodes attached to the temporal and vertex sites (C_{z} and T3 in the 10-20 system). After amplification, the EEG is fed into four parallel band-pass filters with respective upper and lower frequencies set as follows: “delta” (2.4-4.0 Hz), “theta” (4.0-7.5 Hz), “alpha” (7.5-13.5 Hz) and “beta” (13.5-26.0 Hz). Each filter output is sampled 32 times for 5-s periods while the subject is instructed to respond to a series of clicks presented at intervals varying from 8 to 12 s. The output is rectified and averaged to yield the mean rectified voltage in each of the four wavebands. In addition, the four values are summed and each is expressed as a percentage of the total.

The averaged evoked responses are obtained from the 500 ms epoch of the EEG following each of the 32 click stimuli. The averaged response is displayed on an oscilloscope screen and the four peaks (P1, the first positive wave in the 30-60 ms latency range, N1, the first negative wave with a latency of 100-160 ms and N2, the second negative wave with a latency of 130-200 mg) are identified semi-automatically. The latency at each peak and peak-to-peak amplitudes are computed and recorded automatically. Reductions in amplitude and increases in latency are indicators of reduced responsiveness to stimuli and are frequently correlated with subjective decreases in alertness. Conversely, increases in amplitude and/or reductions in latency are objective indicators of increased responsiveness to stimuli.

Skin conductance, blood pressure and pulse rate can be measured by the method of Higgitt et al., supra. See especially page 396, which is incorporated herein by reference in its entirety.

Finger tremor is measured using an accelerometer as discussed by Higgitt et al., supra. An accelerometer is taped to the dorsal surface of the middle finger of the left hand just proximal to the nail bed. The hand is held out with wrist extended and lower arm supported by the arm of the chair. The signal is amplified and 16 5-s samples are frequency analyzed on line using fast Fourier transformation.

Critical flicker fusion threshold is measured using a red LED at the end of a 20 cm black tube according to the method of Higgitt et al, supra. Each subject view the stimulus using his or her dominant eye. The duration of the on-off cycle is changed in 0.5 Hz steps each second. Six alternating ascending and descending trials are administered commencing at 20 and 50 Hz, respectively. The mean of the six limit values is used as the estimate of the threshold.

Key tapping rate is measured per the method of Higgitt et al., supra. The subject is instructed to tap a one inch diameter key as fast as possible for 60 s. The mean inter-tap interval is calculated as a measure of motor speed.

Auditory reaction time is measured to 32 clicks of moderate intensity per Higgitt et al., supra. The mean reciprocal value is calculated.

Paper-and-pencil performance measures, including a cancellation task, a digit symbol substitution test and a symbol copying test are performed essentially as described by Higgitt et al., supra. In the cancellation task, subjects are instructed to cross out all the 4's in a block of numbers containing 40 target items. Time to complete the task and number of errors are recorded.

The digit symbol substitution test (DSST) is a sub-test of the Wechsler Adult Intelligence Scale (WAIS), which assesses coding skills and involves the substitution of symbols for numbers. The task is presented as in the WAIS manual and the measure is the number of correct codings in a 90-s period.

The symbol copying test measures the motor component of the DSST as the subject is instructed to copy the same symbols as are used in the DSST. The score is the number of items correctly copied in a 90-s period. Sixteen equivalent forms of the above three tests are used, one for each time of testing, to minimize practice effects.

Additional tests of cognitive function, such as memory and learning ability, may also be used.

Self ratings of patient mood and bodily symptoms are performed essentially per Higgitt et al., supra.

It is expected that flumazenil-treated Down Syndrome patients will demonstrate an improvement in one or more indicators of cognition as compared to placebo-treated patients.

It is expected that bretazenil-treated Down Syndrome patients will demonstrate an improvement in one or more indicators of cognition as compared to placebo-treated patients.

The foregoing testing may also be applied to mentally retarded patients—e.g. patients having I.Q. scores between about 55 and 70.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating Down Syndrome or mental retardation, comprising administering to a patient suffering from Down Syndrome or mental retardation an effective amount of a composition comprising at least one active pharmaceutical ingredient selected from a benzodiazepine receptor antagonist, a partial benzodiazepine agonist, or both.

2. The method of claim 1, wherein the active pharmaceutical ingredient comprises at least one benzodiazepine receptor antagonist.

3. The method of claim 2, wherein at least one benzodiazepine receptor antagonist is flumazenil.

4. The method of claim 3, wherein the method comprises administering to the patient about 0.05 to about 30 mg of flumazenil one to four times daily, preferably about 0.1 to about 15 mg of flumazenil one to four times daily.

5. The method of claim 1, wherein the active pharmaceutical ingredient comprises at least one partial benzodiazepine agonist.

6. The method of claim 5, wherein at least one partial benzodiazepine agonist is bretazenil.

7. The method of claim 6, wherein the method comprises administering about 0.05 to about 30 mg of bretazenil one to four times daily, preferably about 0.1 to about 15 mg of bretazenil one to four times daily.

8. The method of claim 1, wherein the composition comprising the active pharmaceutical ingredient is an oral, buccal or sublingual composition.

9. The method of claim 8, wherein the composition comprising the active pharmaceutical ingredient is in the form of a tablet, capsule, gel capsule, caplet or liquid solution or suspension.

10. The method of claim 1, wherein the composition comprising the active pharmaceutical ingredient is a parenteral preparation.

11. The method of claim 10, wherein the parenteral preparation is an intravenous injection.

12. The method of claim 1, wherein the effective amount of the composition comprising the active pharmaceutical ingredient is a sub-seizure inducing amount.

13. The method of claim 1, wherein the effective amount of the composition is effective to produce a memory enhancing effect, a learning enhancing effect, or both.

14. A method of enhancing cognitive function in a patient suffering from mental retardation or Down Syndrome, comprising administering to the patient a cognitive function enhancing amount of an active pharmaceutical ingredient comprising a benzodiazepine receptor antagonist, a partial benzodiazepine agonist or both.

15. The method of claim 14, wherein the active pharmaceutical ingredient comprises at least one benzodiazepine receptor antagonist.

16. The method of claim 15, wherein at least one benzodiazepine receptor antagonist is flumazenil.

17. The method of claim 16, wherein the method comprises administering to the patient about 0.05 to about 30 mg of flumazenil one to four times daily, preferably about 0.1 to about 15 mg of flumazenil one to four times daily.

18. The method of claim 14, wherein the active pharmaceutical ingredient comprises at least one partial benzodiazepine agonist.

19. The method of claim 18, wherein at least one benzodiazepine agonist is bretazenil.

20. The method of claim 19, wherein the method comprises administering about 0.05 to about 30 mg of bretazenil one to four times daily, preferably about 0.1 to about 15 mg of bretazenil one to four times daily.

21. The method of claim 1, wherein at least one cognitive function that is enhanced is memory or learning.

22. The method of claim 14, wherein the composition is an oral, buccal or sublingual composition.

23. The method of claim 22, wherein the composition comprising the active pharmaceutical ingredient is in the form of a tablet, capsule, gel capsule, caplet, liquid solution, liquid suspension or fast-dissolve tablet.

24. The method of claim 14, wherein the composition comprising the active pharmaceutical ingredient is a parenteral preparation.

25. The method of claim 24, wherein the composition comprising the active pharmaceutical ingredient is an intravenous injection.

26. The method of claim 14, wherein the effective amount of the composition as a sub-seizure inducing amount.

27. An oral composition for the treatment of mental retardation, Down Syndrome, memory loss or impaired learning, comprising an effective amount of an active pharmaceutical ingredient comprising a benzodiazepine antagonist, a partial benzodiazepine agonist or both.

28. The oral composition of claim 27, wherein the active pharmaceutical ingredient comprises at least one benzodiazepine receptor antagonist.

29. The oral composition of claim 28, wherein at least one benzodiazepine receptor antagonist is flumazenil.

30. The oral composition of claim 29, wherein the composition is in unit dosage form and comprises about 0.05 to about 30 mg of flumazenil, preferably about 0.1 to about 15 mg of flumazenil per dose.

31. The oral composition of claim 28, wherein the active pharmaceutical ingredient consists essentially of a benzodiazepine antagonist.

32. The oral composition of claims 31, wherein the benzodiazepine antagonist is flumazenil.

33. The oral composition of claim 32, wherein the composition is in unit dosage form and comprises about 0.05 to about 30 mg, preferably about 0.1 to about 15 mg of flumazenil per dose.

34. The oral composition of claim 27, wherein the active pharmaceutical ingredient comprises at least one partial benzodiazepine agonist.

35. The oral composition of claim 34, wherein at least one partial benzodiazepine agonist is bretazenil.

36. The oral composition of claim 35, wherein the composition is in unit dosage form an comprises about 0.05 to about 30 mg, preferably about 0.1 to about 15 mg of bretazenil per dose.

37. The oral composition of claim 27, wherein the active pharmaceutical ingredient consists essentially of a partial benzodiazepine agonist.

38. The oral composition of claim 37, wherein the partial benzodiazepine agonist is bretazenil.

39. The oral composition of claim 38, wherein the composition is in unit dosage form an comprises about 0.05 to about 30 mg, preferably about 0.1 to about 15 mg of bretazenil per dose.

40. The oral composition of claim 27 in the form of an oral tablet, caplet, capsule, gel capsule, liquid solution, liquid suspension or fast-dissolve tablet.

41. The oral composition of claim 27, in an extended release, delayed release, pulsatile or controlled release dosage form.

42. The composition of claim 27, wherein the effective amount of the active pharmaceutical ingredient is a sub-seizure inducing amount.

43. The composition of claim 27, wherein the effective amount of the active pharmaceutical ingredient is effective to produce a memory enhancing effect, a learning enhancing effect, or both.

44. A sublingual or buccal composition for the treatment of mental retardation, Down Syndrome, memory loss or impaired learning, comprising an effective amount of an active pharmaceutical ingredient comprising a benzodiazepine antagonist, a partial benzodiazepine agonist or both.

45. The buccal or sublingual composition of claim 44, wherein the active pharmaceutical ingredient comprises at least one benzodiazepine receptor antagonist.

46. The buccal or sublingual composition of claim 45, wherein at least one benzodiazepine receptor antagonist is flumazenil.

47. The buccal or sublingual composition of claim 46, wherein the composition is in unit dosage form and comprises about 0.05 to about 30 mg of flumazenil, preferably about 0.1 to about 15 mg of flumazenil per dose.

48. The buccal or sublingual composition of claim 44, wherein the active pharmaceutical ingredient consists essentially of a benzodiazepine receptor antagonist.

49. The buccal or sublingual composition of claims 48, wherein the benzodiazepine receptor antagonist is flumazenil.

50. The buccal or sublingual composition of claim 49, wherein the composition is in unit dosage form and comprises about 0.05 to about 30 mg, preferably about 0.1 to about 15 mg of flumazenil per dose.

51. The buccal or sublingual composition of claim 44, wherein the active pharmaceutical ingredient comprises at least one partial benzodiazepine agonist.

52. The buccal or sublingual composition of claim 51, wherein at least one partial benzodiazepine agonist is bretazenil.

53. The buccal or sublingual composition of claim 52, wherein the composition is in unit dosage form an comprises about 0.05 to about 30 mg, preferably about 0.1 to about 15 mg of bretazenil per dose.

54. The buccal or sublingual composition of claim 44, wherein the active pharmaceutical ingredient consists essentially of a partial benzodiazepine agonist.

55. The buccal or sublingual composition of claim 54, wherein the partial benzodiazepine agonist is bretazenil.

56. The buccal or sublingual composition of claim 55, wherein the composition is in unit dosage form an comprises about 0.05 to about 30 mg, preferably about 0.1 to about 15 mg of bretazenil per dose.

57. The buccal or sublingual composition of claim 44 in the form of a fast-dissolve tablet or strip.

58. The composition of claim 44, wherein the effective amount of the active pharmaceutical ingredient is a sub-seizure inducing amount.

59. The composition of claim 44, wherein the effective amount of the active pharmaceutical ingredient is effective to produce a memory enhancing effect, a learning enhancing effect, or both.
